(19) United States
(12) Patent Application Publication
(10) Pub. No.: US 2023/0131424 A1
(43) Pub. Date: Apr. 27, 2023

(54) Covenant Pain Relieving Cream

(71) Applicant: Elise Nicks, Milwaukee, WI (US)

(72) Inventor: Elise Nicks, Milwaukee, WI (US)

(21) Appl. No.: 17/451,723

(22) Filed: Oct. 21, 2021

(51) Int. Cl. A61K 36/889; A61K 36/534; A61K 36/61; A61K 36/324; A61K 36/28; A61K 36/185; A61K 36/63; A61K 47/46

(52) U.S. Cl. CPC A61K 36/889; A61K 36/534; A61K 36/61; A61K 36/324; A61K 36/28; A61K 36/185; A61K 36/63; A61K 47/46

(57) ABSTRACT

Use to provide fast pain relief and an instant cooling comfort effect, for people who suffer from arthritis pain, aching joints, anti-inflammatory, antioxidant, foot pain, infections, joint discomfort, muscle aches, muscle soreness, neuropathy pain, nerve conditions, rheumatoid arthritis, stiffness, workout muscle soreness, deep cuts, and soothe foot pain.

### SPECIFICATION

Composition and Methods Used for Pain Control through Natural Oils and Mineral

### BACKGROUND OF THE INVENTION

Covenant Pain Relieving Cream has many uses and benefits. It is made with natural essential oils that provide fast pain relief and an instant cooling comfort effect, when applied to skin.

### BRIEF BACKGROUND OF THE INVENTION

Covenant Pain Relieving Cream can be applied to body, face, hands, knees, elbows, buts or anywhere there is pain.

### DETAILED DESCRIPTION OF THE INVENTION

If you suffer from; arthritis pain, aching joints, anti-inflammatory, antioxidant, foot pain, infections, joint discomfort, muscle aches, muscle soreness, neuropathy pain, nerve conditions, rheumatoid arthritis, stiffness, workout muscle soreness, deep cuts, soothe foot pain. It is also known to help pain in dogs.

This product is sensitive to heat. We recommend storing in a cool, dark, dry place to maintain regularity and promote healing throughout the whole body.

I claim:

1. The present disclosure is directed to a composition intended for application onto human and animal skin suffering from arthritis, aching joints, anti-inflammatory, foot pain, joint discomfort, muscle aches, neuropathy, deep cuts, and workout stiffness; controlled by natural essential oils, the composition comprising (1) a mixture of at least: (a) Coconut Oil cream (b) Peppermint Oil (c) Eucalyptus (d) Frankincense (e) Helichrysum oil (f) Corn mint (g) Olive Oil (h) Hemp Oil (i) Bentonite Clay, (a-i) used together help promote healing throughout the whole body.
